(19) United States
(12) Patent Application Publication
(10) Pub. No.: US 2008/0003606 A1
(43) Pub. Date: Jan. 3, 2008

(54) DIAGNOSIS OF TAMOXIFEN SENSITIVITY

(76) Inventors: Goran Landberg, Limhamn (SE); Karin Jirstrom, Limhamn (SE)

Correspondence Address: GAUTHIER & CONNORS, LLP, 225 FRANKLIN STREET, SUITE 2300, BOSTON, MA, 02110, US

(21) Appl. No.: 11/756,672

(22) Filed: Jun. 1, 2007

Related U.S. Application Data
(63) Continuation of application No. PCT/SE05/01839, filed on Dec. 5, 2005.

(30) Foreign Application Priority Data
Dec. 3, 2004 (SE) 0402947-6

(51) Int. Cl. C12Q 1/68

(52) U.S. Cl. 435/6

(57) ABSTRACT

Data indicates that adjuvant tamoxifen treatment adversely influences outcome in breast cancer with CCND1-amplification and in fact severely promotes disease progress. The present invention thus relates to a method for diagnosing sensitivity to tamoxifen by monitoring the amplification of the gene for cyclin D1, CCND1.

### TECHNICAL FIELD

The present invention relates to a diagnosis of tamoxifen sensitivity, particularly for the post treatment of surgically extracted breast cancer tumours in women.

### BACKGROUND OF THE INVENTION

Loss of normal growth control, including aberrant cell cycle regulation, is one of the hallmarks of cancer. Central in the regulation of the G1/S-transition in the cell cycle is the p16/cyclin D/retinoblastoma protein (pRb)-pathway, which seems to be deregulated in a large fraction of all malignancies, but with a certain degree of cell type specificity.

In breast cancer, overexpression of cyclin D1 is the most common cell cycle aberration, followed by cyclin E overexpression, decreased expression of the p27^{Kip1} cdk inhibitor and silencing of the p16^{Ink4A} gene through promoter methylation. Cyclin D1 overexpression at the mRNA- as well as protein level has been demonstrated in up to 50% of primary breast cancers [1-3] with amplification of its corresponding gene CCND1 in about 15% [4]. The further existence of a cross talk between cyclin D1 and the estrogen receptor (ER) machinery is predominantly observed in ER positive, more well-differentiated breast cancers [5, 6]. 17-β Estradiol (E2) also induces cyclin D1 gene expression in ER positive breast cancer cell lines and cyclin D1 can bind to the ER and the co-factor SRC-1 and potentially activate the receptor without any ligand binding.

While cyclin D1 is induced early in the G1 phase (Gap-phase 1 before DNA-synthesis) and plays a critical role for progression of cells through G1, cyclin A2 accumulates during S-phase (DNA-synthesis phase) and functions during S-phase and at the G2/M transition. Cyclin A2 may, similarly to cyclin D1, also affect the ER and via activation of cdk2 induce phosphorylation of serines 104/106 of ER, potentially inducing ligand independent activation. Thus, besides central roles in cell cycle regulation, cyclin D1 and cyclin A2 seems to be able to directly influence the ER and potentially modify its response to estrogens and anti-estrogens.

Antiestrogens are the treatment of choice for hormone-dependent breast cancer and it has been known for many years that the nonsteroidal antiestrogens, among which tamoxifen is prototypic, arrest cells in early G1 phase. Today, the majority of all ER-positive breast cancer will receive adjuvant antiestrogen treatment and as shown in many studies, tamoxifen substantially improves patient survival [7, 8].

It is also clear that a large fraction of patients do not respond as expected to tamoxifen treatment despite having ER-positive tumors. Some patients may even have an adverse outcome, due to a potential ER-agonistic effect of tamoxifen under certain conditions.

In line with a direct interaction of cyclin D1 and A2 on the ER, these proteins may be involved in tamoxifen resistance in breast cancer. A reduction in cyclin D1 mRNA and protein expression has also been demonstrated as an early and critical event in antiestrogen action in vitro [9] and short-term ectopic induction of cyclin D1 expression in ER-positive cell lines has been demonstrated to overcome antiestrogen induced inhibition of cell cycle progression [10]. Gene expression analyses show that cyclin A2 is induced in response to estrogen as well as tamoxifen treatment, while cyclin D1 is not induced by tamoxifen but constitutively expressed in tamoxifen resistant cells [11]. In one study, high cyclin A2 expression has been associated with an impaired tamoxifen response [12] but the predictive value has not yet been investigated in a randomized trial.

It has previously been demonstrated an impaired tamoxifen response in postmenopausal women with highly ER positive and cyclin D1 overexpressing breast cancer, in a randomized trial with long-term follow up. Interestingly, the intensity of the nuclear staining by immunohistochemistry rather than the nuclear fraction was indicative of treatment response. The reason for this discrepancy remains to be elucidated but the nuclear intensity of cyclin D1 might be linked to the degree of amplification of the CCND1 gene [13].

### SUMMARY OF THE PRESENT INVENTION

The present invention relates to the mapping of tamoxifen sensitivity among in particular ER positive breast cancer treated women, either pre or post menopausal. The breast cancer treatment as such being of non-importance as such, and the treatments are normally surgical, cytotoxic and/or radiation. The aim is thereby to avoid tamoxifen treatment in a certain group of women thereby avoiding negative, adverse effects of tamoxifen due to its agonistic effects.

### DETAILED DESCRIPTION OF THE PRESENT INVENTION

The present findings have led to the present invention which relates to the testing of a tissue sample with respect to identification of the cyclin D1 expressing gene, CCND1, as any amplification of said gene is found to be an expression for negative sensitivity to tamoxifen treatment. The invention is thus related to the diagnosis of tamoxifen women, which further are node positive.

In an attempt to define subgroups of breast cancer that respond differently to tamoxifen treatment immunohistochemical cyclin D1 and A2 expression was focused on by high throughput tissue analyzes of 500 premenopausal breast cancer samples included in a randomized trial with long-term follow-up. By comparing the untreated control patients with patients receiving tamoxifen, subgroups that responded differently could be characterized. In addition, FISH-analyses of CCND1 gene amplification were performed in order to explore a possible relationship with the localization and staining intensity of the cyclin D1 protein and also to investigate whether the presence or absence of gene amplification has an independent predictive value for tamoxifen response.

Material and Methods

Patient Material

During 1984-1991, 564 premenopausal patients or patients under 50 years with stage 11 (pT2 N0 M0, pT1 N1 M0 and pT2, N1 M0) invasive breast cancer were enrolled in a randomized trial of 2 years of adjuvant tamoxifen treatment with a daily dosage of either 40 mg (study center 1) or 20 mg (study center 2) or no adjuvant treatment (detailed in [14]). Less than two percent of the patients (n=9) received adjuvant polychemotherapy. The median follow-up for patients without breast cancer event was 13.9 years (95% CI: 13.6-14.3).

Tissue Microarray Construction

All available archived primary tumor specimens were collected (n=500). Areas with invasive cancer were marked on the H&E stained slides and two 0.6 mm tissue cores were taken from each donor block and mounted in a recipient block using an automated arrayer ((ATA-27, Beecher Inc, WI, USA).

Immunohistochemistry:

For immunohistochemistry, 4 μm sections were dried, deparaffinized, rehydrated and microwave treated for 2×5 minutes in a citrate buffer (pH of 6.0) before being processed in an automatic immunohistochemistry staining machine (Techmate 500, DAKO, Copenhagen, Denmark) using the monoclonal antibody cyclin A2 (H432, 1:200, Santa Cruz, Calif., USA) and cyclin D1 (Clone DSC-6, 1:100, DAKO A/S, Glostrup, Denmark).

For cyclin A2 expression, only the fraction of positively staining nuclei were calculated (405 cases) and subdivided into five groups based on the following score: 0 (0-1%), 1 (2-10%), 2 (11-25%), 3 (26-50%) and 4 (>50%). For cyclin D1, the nuclear fraction and intensity as well as the cytoplasmic staining intensity was evaluated (463 cases). The nuclear fractions were divided into the following five subgroups: 0 (0%), 1 (1-25%), 2 (26-50%), 3 (51-75%) and 4 (>75%). The nuclear and cytoplasmic intensity was evaluated as absent=0, weak=1, intermediate=2 and strong=3. The TMA:s had previously been analyzed immunohistochemically for estrogen- (ER) and progesterone (PR) receptor status (anti-ER Clone 6F11 and anti-PgR Clone 16) using the Ventana Benchmark system (Ventana Medical Systems Inc., AZ, USA). In line with the clinically established cut-off used for hormone receptor assessment, tumors with more than 10% positively staining nuclei were considered positive. Ki-67 index had also been assessed previously using the monoclonal antibody Ki-67 (1:200, M7240, DAKO, Denmark).

Fluorescence In Situ Hybridization:

For fluorescence in situ (FISH) analysis of CCND1 gene amplification, two direct-labeled probes were used, LSI cyclin D1 (11q13) SpectrumOrange against CCND1 (11q13) and CEP 11 SpectrumGreen (Vysis Inc., Illinois, USA) against the centromere of chromosome 11. The FISH analysis was performed according to the standard protocol recommended by the manufacturer and according to “LSI® Locus Specific Identifier DNA probes” from Vysis Inc., Illinois. Briefly, tissue sections were de-paraffinized in xylene and alcohol and air-dried. The slides were then microwave-treated in Target Retrieval Solution pH 7.3 (DAKO A/S, Glostrup, Denmark) for 5+5 minutes and treated with 100 μl pepsin (Digest-All 3, Zymed, California, USA) for 8 minutes in 37° C. and washed in water. The de-paraffinized slides were denatured in 70% formamide/2×SSC at 73° C. for 5 minutes followed by dehydration in graded ethanol. A denatured mixture of 1 μl LSI probe, 2 μl pH₂O and 7 μl LSI hybridization buffer was added on each slide and incubated at 37° C. overnight. The slides were then washed with 0.4×SSC/0.3% Nonidet P40 at 37±1° C. for 2 minutes followed by a 2×SSC/0.1% NP40 wash at ambient temperature for 1 minute to remove non-specifically bound probe.

For evaluation, the CCND1 gene was considered amplified when the ratio of orange/green was >1. Non-amplified cases were classified as 0, cases with up to 10 copies as 1 and >10 copies as 2. CCND1 gene status could be assessed in 280 cases (56% of the present study and (49.6 of the original). 44 cases (15%) were amplified (>=3 copies), 10 of these (2.8%) with a copy number >10. Apart from the 64 missing tumors, CCND1 status could not be determined in 220 tumors in the TMA:s, despite repeated analyses on consecutive sections. A subset of 50 tumors without signal were analyzed in a separate, manually constructed TMA, using 1.0 mm cores but this approach did not increase the amount of valid cases.

Statistics

Baseline prognostic and patient characteristics between the original study group, patients with available FISH data (n=280) and patients without FISH data (n=284) were compared using the chi-square test to exclude the possibility of selection bias. Baseline prognostic and patients characteristics were also compared between amplified and non-amplified cases using the same approach as well as the marker distribution according to trial arm. For evaluation of treatment response, only tumors with >10% ER positivity were included. Kaplan-Meier and log-rank tests and univariate Cox regression analyses were used for recurrence free survival (RFS), breast cancer survival (BCS) and overall survival (OS). RFS considered local, regional, distant recurrences and breast-cancer specific death, but not contralateral breast cancer, as primary event. The interaction between tamoxifen treatment and the investigated parameters was further explored by a Cox model including one of the four variables respectively, a treatment variable and an interaction-variable. All statistical tests were two-sided. Calculations were performed with SPSS 11.0 (SPSS inc., Chicago, Ill., USA).

Results

Cyclin D1, A2 and Clinicopathological Parameters

Examples of immunohistochemical staining of cyclin D1 and A2 as well as FISH-analyses of CCND1 copy numbers are illustrated in FIG. 1 a-d and distribution according to important patient and tumor characteristics in table 1 with ER-positive cases in brackets and according to trial arm in table 2. As expected, cyclin D1 protein overexpression was more frequent in ER-positive tumors and cyclin A2 in ER-negative tumors. Cyclin D1 nuclear fraction, -intensity and cytoplasmic intensity all correlated strongly with each other (not shown) and cyclin D1 was negatively associated with proliferation except for in the subgroup of ER-positive tumors, confirming a positive correlation between cyclin D1 and proliferation. Cyclin A2, was overall positively associated with Ki-67, histological grade (NHG) and inversely associated with PR status while cyclin D1 protein expression and amplification were positively associated with PR. CCND1 gene amplification further correlated significantly with cyclin D1 protein expression, but not to cyclin A2. The majority of amplified tumors (42/44=(95.5%) were ER positive. Baseline clinico pathological and patient characteristics according to trial arm in cases with and without CCND1 status are demonstrated in table 3. The relationship of clinico pathological parameters as well as immunohistochemical marker expression in relation to CCND1 amplification status is demonstrated in table 4.

Above the amplication has been determined using FISH. However, other methods such as other ISH methods are useable as well.

TABLE 1
Association between cyclin A2 and D1 protein expression, CCND1 amplification and important clinicopathological variables.*

| | | | | | |
|---|---|---|---|---|---|
| Variable | D1nf (ER+) | D1ni (ER+) | D1ci (ER+) | A2nf (ER+) | CCND1amp (ER+) |

Age

| SCC | 0.007 | (−0.01) | 0.13 | (0.04) | 0.04 | (−0.004) | −0.10 | (−0.13) | 0.05 | (0.04) |
|---|---|---|---|---|---|---|---|---|---|---|
| p | 0.09 | (0.80) | 0.004** | (0.43) | 0.37 | (0.94) | 0.05*/0.03* | | 0.36 | (0.59) |
| n | 463 | (312) | 463 | (312) | 463 | (315) | 405/ | (263) | 280 | (193) |

Nodes +/−

| SCC | 0.10 | (−0.01) | 0.11 | (0.04) | 0.03 | (−0.02) | −0.01 | (0.10) | 0.08 | |
|---|---|---|---|---|---|---|---|---|---|---|
| p | 0.04* | (0.88) | 0.02* | (0.43) | 0.53 | (0.74) | 0.77 | (0.12) | 0.18 | |
| n | 461 | (311) | 461 | (311) | 464 | (314) | 403 | (262) | 279 | (193) |

Size

| | | | | | | | | | | |
|---|---|---|---|---|---|---|---|---|---|---|
| SCC | −0.11 | (−0.04) | −0.17 | (−0.14) | 0.04 | (−0.07) | −0.10 | (−0.01) | −0.08 | (−0.08) |
| p | 0.02* | (0.45) | <0.001** | (0.01*) | 0.37 | (0.22) | 0.05* | (0.82) | 0.16 | (0.25) |

| | | | | | | | | | |
|---|---|---|---|---|---|---|---|---|---|
| n | 462 | (311) | 462 | (311) | 466/314 | 405 | (262) | 280 | (193) |

NHG

| | | | | | | | | | | |
|---|---|---|---|---|---|---|---|---|---|---|
| SCC | −0.23 | (0.05) | −0.23 | (0.06) | −0.23 | (−0.11) | 0.47 | (0.30) | −0.08 | (0.06) |
| p | <0.001** | (0.40) | <0.001** | (0.30) | <0.001** | (0.04*) | <0.001** | (<0.001**) | 0.17 | (0.40) |
| n | 450 | (310) | 450 | (310) | 453 | (313) | 392 | (260) | 273 | (192) |

Ki67

| | | | | | | | | | | |
|---|---|---|---|---|---|---|---|---|---|---|
| SCC | −0.153 | (0.202) | −0.14 | (0.26) | −0.07 | (0.09) | 0.72 | (0.64) | −0.02 | (0.09) |
| p | 0.002** | (0.001**) | 0.004** | (<0.001**) | 0.13 | (0.12) | <0.001** | (<0.001**) | 0.74 | (0.24) |
| n | 407 | (272) | 407 | (272) | 410 | (275) | 393 | (255) | 257 | (178) |

PR

| | | | | | | | | | | |
|---|---|---|---|---|---|---|---|---|---|---|
| SCC | 0.42 | (0.11) | 0.42 | (0.07) | 0.38 | (0.19) | −0.35 | (−0.13) | 0.19 | (0.08) |
| p | <0.001** | (0.06) | <0.001 | (0.23) | <0.001** | (0.001**) | <0.001** | (0.03*) | 0.002** | (0.58) |
| n | 435 | (295) | 435 | (298) | 438 | (298) | 387 | (254) | 266 | (187) |

A2nf

| | | | | | | | | |
|---|---|---|---|---|---|---|---|---|
| SCC | −0.04 | (0.27) | −0.03 | (0.32) | −0.07 | (0.03) | — | −0.02 |
| p | 0.45 | (<0.001**) | 0.54 | (<0.001**) | 0.20 | (0.65) | — | 0.70 |
| n | 386 | (255) | 386 | (255) | 389 | (258) | — | 250 |

D1amp

| | | | | | | | | | |
|---|---|---|---|---|---|---|---|---|---|
| SCC | 0.38 | (0.33) | 0.36 | (0.30) | 0.15 | (0.09) | −0.02 | (0.06) | — |
| p | <0.001** | (<0.001) | <0.001** | (<0.001) | 0.01* | (0.23) | 0.69 | (0.41) | — |
| n | 275 | (191) | 277 | (191) | 277 | (193) | 250 | (173) | — |

SCC = Spearman's Correlation Coefficient

TABLE 2
Cyclin D1 and A2 distribution according to trial arm

| | | |
|---|---|---|
| Tamoxifen | Control | |
| (ER+) | (ER+) | p-value* |

D1 Nuclear fraction

| | | | | | |
|---|---|---|---|---|---|
| 0% | 56 | (10) | 53 | (13) | 0.55 |
| 1-25% | 69 | (46) | 91 | (70) | (0.35) |
| 26-50% | 58 | (50) | 52 | (43) | |
| 51-75% | 32 | (31) | 36 | (33) | |
| >75% | 8 | (8) | 8 | (8) | |

D1 Nuclear intensity

| | | | | | |
|---|---|---|---|---|---|
| 0 | 56 | (10) | 53 | (13) | 0.38 |
| 1 | 69 | (48) | 89 | (63) | (0.48) |
| 2 | 73 | (63) | 79 | (73) | |
| 3 | 25 | (24) | 19 | (18) | |

D1 cytoplasmic intensity

| | | | | | |
|---|---|---|---|---|---|
| 0 | 20 | (3) | 12 | (5) | 0.34 |
| 1 | 85 | (48) | 91 | (49) | (0.78) |
| 2 | 81 | (60) | 85 | (65) | |
| 3 | 37 | (34) | 52 | (48) | |
| 3+++ | 1 | (1) | 2 | (2) | |

CCND1 amplification

| | | | | | |
|---|---|---|---|---|---|
| 0 | 111 | (72) | 125 | (79) | 0.80 |
| 1 | 18 | (16) | 16 | (16) | (0.96) |
| 2 | 5 | (5) | 5 | (5) | |

A2 Nuclear fraction

| | | | | | |
|---|---|---|---|---|---|
| 0-1% | 24 | (12) | 28 | (22) | 0.94 |
| 2-10% | 75 | (57) | 82 | (68) | (0.42) |
| 11-25% | 69 | (45) | 71 | (46) | |
| 26-50% | 27 | (7) | 24 | (5) | |
| 51-100% | 3 | (1) | 2 | (−) | |

*Chi square

TABLE 3
Baseline characteristics of patients
with known and unknown CCND1 status

| | | | |
|---|---|---|---|
| | CCND1 status | CCND1 status | |
| | known | unknown | |
| Category | n = 280(%) | n = 284(%) | p-value |

Age (years)

| | | | |
|---|---|---|---|
| median(range) | 44(25-57) | 45(27-55) | 0.16 [symbol] |
| <40 | 59(21.1) | 54 | 0.34 [symbol] |
| 40-49 | 183(65.3) | 179 | |
| 50- | 38(13.6) | 51 | |

Tumour size

| | | | |
|---|---|---|---|
| median (range) | 23(2-50) | 25(2-75) | 0.47 [symbol] |
| <20 mm | 113(40.3) | 95(33.4) | 0.10 [symbol] |
| >20 mm | 167(59.6) | 188(66.2) | |
| unknown | | 1(0.4) | |

Node status

| | | | |
|---|---|---|---|
| 0 | 75(26.8) | 85(29.9) | 0.81 [symbol] |
| 1-3 | 142(50.7) | 133(46.8) | |
| >4 | 62(22.1) | 65(22.9) | |
| unknown | 1(0.4) | 1(0.4) | |

NHG

| | | | |
|---|---|---|---|
| 1 | 36(12.9) | 22(7.7) | 0.35 [symbol] |
| 2 | 116(41.4) | 106(37.3) | |
| 3 | 121(43.2) | 113(39.8) | |
| unknown | 7(2.5) | 43(15.1) | |

ER

| | | | |
|---|---|---|---|
| negative | 81(28.9) | 70(24.6) | 0.26 [symbol] |
| positive | 193(68.9) | 131(46.1) | |
| not evaluated | 6(2.1) | 83(29.2) | |

PR

| | | | |
|---|---|---|---|
| negative | 87(31.1) | 76(26.8) | 0.14 [symbol] |
| positive | 179(63.9) | 115(40.5) | |
| not evaluated | 14(5.0) | 93(32.7) | |

Ki-67 index

| | | | |
|---|---|---|---|
| 0-1% | 38(13.6) | 24(8.4) | 0.19 [symbol] |
| 2-10% | 89(31.8) | 43(15.1) | |
| 10-25% | 68(24.3) | 50(17.6) | |
| 25-50% | 30(10.7) | 28(9.9) | |
| >50% | 32(11.4) | 28(9.9) | |
| not evaluated | 23(8.2) | 111(39.1) | |

[symbol]

Mann-Whitneys u-test for comparison of medians [symbol]
Chi Square test for kx2 tables

TABLE 4
Clinicopathological characteristics and marker distribution according to CCND1
amplification status for all and estrogen receptor (ER) positive tumours.

| | | | | | |
|---|---|---|---|---|---|
| CCND1 | CCND1 | | CCND1 | CCND1 | |
| amplified | non-amplified | | amplified ER+ | non-amplified ER+ | |
| n = 44(%) | n = 236(%) | p-value* | n = 42(%) | n = 151(%) | p-value* |

Age (years)

| | | | | | | |
|---|---|---|---|---|---|---|
| median(range) | 44.5(33-52) | 44(25-57) | 0.81 | 44.5(33-52) | 45(26-57) | 0.78 |
| <40 | 7(15.9) | 52(22.0) | 0.53 | 6(14.3) | 27(17.9) | 0.59 |
| 40-49 | 32(72.7) | 151(64.0) | | 31(73.8) | 99(65.6) | |
| 50- | 5(11.4) | 33(13.0) | | 5(11.9) | 25(16.5) | |

Randomization

| | | | | | | |
|---|---|---|---|---|---|---|
| Tamoxifen | 23(52.3) | 111(47.0) | 0.63 | 21 | 72(47.7) | 0.79 |
| Control | 21(47.7) | 125(53.0) | | 21 | 79(52.3) | |

Tumour size

| | | | | | | |
|---|---|---|---|---|---|---|
| median (range) | 20.5(4-50) | 25(2-55) | 0.11 | 20(4-50) | 23(7-50) | 0.32 |
| <20 mm | 22(50.0) | 91(38.6) | 0.21 | 22(5.5) | 64(42.4) | 0.25 |
| >20 mm | 22(50.0) | 145(61.4) | | 20(47.6) | 87(57.6) | |

Node status

| | | | | | | |
|---|---|---|---|---|---|---|
| 0 | 8(18.2) | 67(28.4) | 0.02 | 8(19.0) | 32(21.2) | 0.42 |
| 1-3 | 28(63.6) | 114(48.3) | | 27(64.3) | 81(53.6) | |
| >4 | 7(15.9) | 55((23.3) | | 7(16.7) | 38(25.2) | |
| not evaluated | 1(2.3) | 2 | | — | — | |

NHG

| | | | | | | |
|---|---|---|---|---|---|---|
| 1 | 2(4.5) | 34(14.4) | 0.001 | 2(4.8) | 30(19.9) | 0.03 |
| 2 | 30(68.2) | 86(36.4) | | 29(69.0) | 75(49.7) | |
| 3 | 12(27.3) | 109(46.2) | | 11(26.2) | 45(29.8) | |
| not evaluated | — | 7(3.0) | | — | 1(0.6) | |

ER

| | | | | | | |
|---|---|---|---|---|---|---|
| negative | 1(2.3) | 80(33.9) | <0.001 | — | — | — |
| positive | 42(95.4) | 151(64.0) | | — | — | |
| not evaluated | 1(2.3) | 5(2.1) | | — | — | |

PR

| | | | | | | |
|---|---|---|---|---|---|---|
| negative | 5(11.4) | 82 | 0.001 | 3(7.1) | 14(9.3) | 0.89 |
| positive | 38(86.4) | 141 | | 38(90.5) | 132(87.4) | |
| not evaluated | 1(2.3) | 13(5.5) | | 1(2.4) | 5(3.3) | |

Ki-67 index

| | | | | | | |
|---|---|---|---|---|---|---|
| 0-1% | 2(4.5) | 36(15.2) | 0.03 | 2(4.8) | 24(15.9) | 0.27 |
| 2-10% | 21(47.8) | 68(28.8) | | 20(47.6) | 57(37.8) | |
| 10-25% | 15(34.1) | 53(22.4) | | 15(35.7) | 35(23.2) | |
| 25-50% | 4(9.1) | 26(11.0) | | 4(9.5) | 16(10.6) | |
| >50% | 2(4.5) | 30(12.7) | | 1(2.4) | 4(2.6) | |
| not evaluated | — | 23(9.7) | | — | 15(9.9) | |

D1 nuclear fraction

| | | | | | | |
|---|---|---|---|---|---|---|
| 0% | 0 | 56(23.7) | <0.001 | 0 | 10(6.6) | <0.001 |
| 1-25% | 9(20.4) | 100(42.4) | | 9(21.4) | 71(47.0) | |
| 26-50% | 15(34.1) | 49(20.8) | | 14(33.13) | 43(28.5) | |
| 51-75% | 11(25.0) | 23(9.7) | | 11(26.2) | 21(13.9) | |
| >75% | 8(18.2) | 4(1.7) | | 8(19.0) | 4(2.6) | |
| not evaluated | 1(2.3) | 4(1.7) | | — | 2(1.3) | |

D1 nuclear intensity

| | | | | | | |
|---|---|---|---|---|---|---|
| 0 | 0 | 56(23.7) | <0.001 | 0 | 10(6.6) | <0.001 |
| 1 | 8(18.2) | 91(38.6) | | 8(19.0) | 61(40.4) | |
| 2 | 22(50.0) | 70(29.7) | | 21(50.0) | 64(42.4) | |
| 3 | 13(29.5) | 15(6.3) | | 13(31.0) | 14(9.3) | |
| not evaluated | 1(2.3) | 4(1.7) | | — | 2(1.3) | |

D1 cytoplasmic intensity

| | | | | | | |
|---|---|---|---|---|---|---|
| 0 | 1(2.3) | 16(6.8) | 0.04 | 0 | 2(1.3) | 0.31 |
| 1 | 13(29.5) | 96(40.7) | | 13(31.0) | 50(33.1) | |
| 2 | 14(31.8) | 83(35.2) | | 14(33.3) | 65((43.1) | |
| 3 | 15(34.1) | 37(15.7) | | 15(35.7) | 32(21.2) | |
| 3+++ | 0 | 2(0.8) | | 0 | 2(1.3) | |
| not evaluated | 1(2.3) | 2(0.8) | | — | — | |

A2 nuclear fraction

| | | | | | | |
|---|---|---|---|---|---|---|
| 0-1% | 1(2.3) | 30(12.7) | 0.04 | 1(2.4) | 21(13.9) | 0.14 |
| 2-10% | 23(52.3) | 78((33.0) | | 22(52.4) | 61(40.4) | |
| 11-25% | 16(36.3) | 75(31.8) | | 16(38.1) | 46(30.5) | |
| 26-50% | 1(2.3) | 24(10.2) | | 0 | 5(3.3) | |
| 51-100% | 0 | 2(0.8) | | 0 | 1(0.7) | |
| not evaluated | 3(6.8) | 27(11.4) | | 3(7.1) | 17(11.2) | |

*Chi Square for kx2 tables and Mann-Whitneys u-test for comparison of medians.

Cyclin D1, A2 and Tamoxifen Response

In line with our previous findings of postmenopausal women, tumors with strong nuclear cyclin D1 staining intensity did not respond to tamoxifen treatment regarding recurrence free survival (RFS), in contrast to tumors with absent to moderate staining intensity as illustrated by Cox univariate regression analysis in table 5 and in the Kaplan Meier curves in FIG. 2b. Treatment response was then tested for the different nuclear fractions of cyclin D1 and A2. For cyclin D1, there was no obvious difference between the subgroups whereas tumors with less than 10% positive cyclin A2 nuclei responded well to tamoxifen in contrast to a lack of response in tumors with >10% positive nuclei (FIG. 2 and table 5). None of the described differences for cyclin D1 and cyclin A2 protein content were nevertheless significant in multivariate interaction analyses as detailed in table 6.

TABLE 5
Recurrence free and breast cancer survival by Cox univariate analyses (ER+)

| | Recurrence Free Survival | | | Overall Survival | | |
|---|---|---|---|---|---|---|
| Category | RR | 95% Cl | p-value | RR | 95% Cl | p-value |

Cyclin D1 nf <25%

| | | | | | | | |
|---|---|---|---|---|---|---|---|
| control | n = 83 | 1.00 | | | 1.00 | | |
| tamoxifen | n = 56 | 0.62 | 0.37-1.04 | 0.07 | 0.65 | 0.37-1.14 | 0.14 |

Cyclin D1 nf >25%

| | | | | | | | |
|---|---|---|---|---|---|---|---|
| control | n = 84 | 1.00 | | | 1.00 | | |
| tamoxifen | n = 88 | 0.61 | 0.38-0.96 | 0.03 | 0.78 | 0.48-1.25 | 0.30 |

Cyclin D1 ni low/intermediate

| | | | | | | | |
|---|---|---|---|---|---|---|---|
| control | n = 149 | 1.00 | | | 1.00 | | |
| tamoxifen | n = 120 | 0.59 | 0.41-0.85 | 0.004 | 0.72 | 0.50-1.06 | 0.10 |

Cyclin D1 ni high

| | | | | | | | |
|---|---|---|---|---|---|---|---|
| control | n = 18 | 1.00 | | | 1.00 | | |
| tamoxifen | n = 24 | 0.84 | 0.30-2.31 | 0.73 | 0.77 | 0.27-2.19 | 0.62 |

Cyclin A2 nf <10%

| | | | | | | | |
|---|---|---|---|---|---|---|---|
| control | n = 51 | 1.00 | | | 1.00 | | |
| tamoxifen | n = 52 | 0.51 | 0.30-0.86 | 0.01 | 0.72 | 0.42-1.25 | 0.25 |

Cyclin A2 nf >10%

| | | | | | | | |
|---|---|---|---|---|---|---|---|
| control | n = 90 | 1.00 | | | 1.00 | | |
| tamoxifen | n = 69 | 0.86 | 0.50-1.47 | 0.59 | 0.92 | 0.53-1.59 | 0.75 |

CCND 1 not amplified

| | | | | | | |
|---|---|---|---|---|---|---|
| control | 1.00 | | | 1.00 | | |
| tamoxifen | 0.39 | 0.23-0.65 | <0.0001 | 0.43 | 0.24-0.76 | 0.004 |

CCND1 amplified

| | | | | | | |
|---|---|---|---|---|---|---|
| control | 1.00 | | | 1.00 | | |
| tamoxifen | 2.22 | 0.94-5.26 | 0.06 | 2.13 | 0.89-5.10 | 0.09 |

TABLE 6
Multivariate Cox proportional hazards model for cyclin A2/D1 and treatment interaction*

| | | Recurrence free survival | | | Overall survival | | |
|---|---|---|---|---|---|---|---|
| Variable | | RR | 95% Cl | p | RR | 95% Cl | p |
| D1 NF | low vs high | 1.00 | 0.63-1.58 | 1.00 | 0.90 | 0.55-1.47 | 0.67 |
| Treatment | tamoxifen vs control | 0.62 | 0.36-1.13 | 0.12 | 0.67 | 0.36-1.24 | 0.20 |
| Interaction variable | tamoxifen × D1 nf | 0.98 | 0.46-2.07 | 0.95 | 1.29 | 0.58-2.87 | 0.53 |
| D1 NI | low/moderate vs high | 0.75 | 0.32-1.74 | 0.50 | 0.94 | 0.40-2.21 | 0.89 |
| Treatment | tamoxifen vs control | 0.62 | 0.42-0.92 | 0.02 | 0.78 | 0.51-1.18 | 0.23 |
| Interaction variable | tamoxifen × D1 ni | 1.27 | 0.39-4.10 | 0.69 | 1.00 | 0.31-3.27 | 0.99 |
| A2 NF | low vs high | 0.95 | 0.55-1.64 | 0.86 | 1.07 | 0.60-1.90 | 0.81 |
| Treatment | tamoxifen vs control | 0.46 | 0.27-0.80 | 0.006 | 0.64 | 0.36-1.13 | 0.13 |
| Interaction variable | tamoxifen × A2 nf | 1.93 | 0.89-4.20 | 0.10 | 1.40 | 0.63-3.13 | 0.41 |
| CCND1 | amplified vs nonamplified | 0.59 | 0.27-1.27 | 0.18 | 0.83 | 0.37-1.83 | 0.64 |
| Treatment | tamoxifen vs control | 0.37 | 0.21-0.64 | <0.001 | 0.41 | 0.21-0.80 | 0.009 |
| Interaction variable | tamoxifen × CCND1 | 6.26 | 2.20-17.90 | 0.001 | 5.85 | 1.93-17.75 | 0.002 |

*Adjusted for age, tumor size, NHG, Ki-67 and nodal status

In this study, CCND1-amplification status was by far the most powerful predictor of tamoxifen response but surprisingly indicated an adverse tamoxifen effect in amplified tumors. For patients with non-amplified tumors, the proportional 10-year recurrence free survival (RFS) was 74% in the treatment arm compared to 44% in the control group and 78% vs 61% for overall survival (OS). For patients with amplified tumors, 10 years RFS was 62% in the untreated versus 29% in the tamoxifen treated arm and the corresponding proportions for OS 74% versus 56%. The tamoxifen response for RFS and OS in non-amplified tumors was highly significant in univariate Cox regression analysis (table 5), while treatment response in amplified tumors was almost significantly adverse for RFS (p=0.06). In multivariate interaction analysis, a significant interaction between tamoxifen treatment and CCND1 amplification was observed both for RFS and OS (table 6).

Since the number of node positive patients was rather high in this study (402/564=71%), we also explored tamoxifen response within this group. Interestingly, in addition to a significant tamoxifen effect in non-amplified tumors, the adverse effect upon treatment was highly significant in amplified tumors (FIG. 3) and 10 year RFS was 68% in the untreated compared to 13% in the treated arm. Analogue to this, the multivariate interaction variable was even more significant for node positive patients with more then nine-fold difference in treatment response for CCND1-amplified tumors (?).

A puzzling finding was that tumors with CCND1-amplification had an adverse tamoxifen effect whereas high cyclin D1 protein intensity defined a group with no tamoxifen effect and a high fraction of cyclin D1 positive cells did not discriminate any treatment differences despite strong associations between all these parameters. In an attempt to address this we further defined subgroups of cyclin D1 alterations. Interestingly, CCND1-amplified tumors without high cyclin D1 protein intensity or >50% cyclin D1 positive cells showed a clear adverse tamoxifen effect despite not being defined as cyclin D1 protein overexpressing tumors. The few tumors with high cyclin D1 protein intensity but no CCND1 gene amplification was not conclusive regarding tamoxifen effects, due to very few events in this subgroup.

Prognostic Information of Cyclin D1 and Cyclin A2

High cyclin A2 expression was associated with worse outcome in the untreated group (RR 1.34 [0.91-1.96]p=0.13 for RFS and RR 1.66[1.11-2.48]p=0.01 for OS) which is line with earlier studies [12, 15] and with proliferation markers in general. In contrast, cyclin D1 protein content (data not shown) or CCND1 amplification (FIG. 4) were not associated with prognosis.

By high throughput tissue microarray analyses of tumors from premenopausal women included in a randomized adjuvant tamoxifen trial, it is clearly demonstrated that CCND1-gene amplification, a non-random genetic alteration occurring in about 15% of all breast cancer, characterizes tumors in which tamoxifen has an agonistic rather than antagonistic effect. As illustrated by the multivariate interaction analyses, patients with CCND1-amplified tumors had a 6-9 fold difference in tamoxifen treatment effect, which is statistically highly significant and clearly strengthens our observations. Furthermore, this remarkable adverse effect was obtained after only 2 years of adjuvant tamoxifen treatment, implicating the possibility of even greater recoil by the standard 5 year treatment used today.

Cyclin D1 protein expression correlated strongly with CCND1-gene amplification, but although indications of an impaired tamoxifen response could be demonstrated for cyclin D1 protein overexpressing tumors, this was in stark contrast to CCND1-amplified tumors, even without an apparent protein overexpression. These findings indicate that the cyclin D1 protein may not primarily be involved in the altered tamoxifen response. Nevertheless, cyclin D1 is strongly linked to the ER and experimental data supports a direct interaction between cyclin D1 and ER [1, 2]. In addition, assessment of cyclin D1 overexpression by FISH-analysis is probably a less subjective and therefore more reliable detection method than evaluation of the immunohistochemical staining intensity. The fraction of cyclin D1 positive cells could further be influenced by the fraction of actively cycling cells of a tumor and might be an even less adequate assessment of cyclin D1 overexpression.

In this study, amplification data could not be retrieved in 44% of the analyzed tumors. Some of these missing data were due to lost tissue cores in the TMAs but the majority could not be retrieved in consecutive sections or manually constructed arrays with larger core diameter. This suggests that a fraction of the tumors could not be analyzed by FISH-technology probably due to not yet standardized laboratory procedures at the time when the tumors in this study were processed (1984-91). However, the group without amplification data did not differ from the analyzed group regarding important tumor characteristics assuring the absence of selection bias in the study.

The CCND1-gene is located at chromosome11q13, a gene-dense region that seems to be amplified in a variety of human malignancies. In this study, the frequency of CCND1 amplification was 15%, which is in concordance with previously reported rates. In breast cancer, the two most eligible key oncogenes on this amplicon are cyclin D1 and EMS1 [4], the latter encoding the human homologue of the cytoskeletal actin-binding protein and c-Src substrate Cortactin. CCND1 and EMS1 amplification seem to confer different phenotypes in ER positive and negative breast cancer. In contrast to cyclin D1, EMS1 overexpression generally results from gene amplification [16], is independent of cyclin D1 and ER expression and is not regulated by estrogen. EMS1 amplification has been associated with early relapse in lymph node negative and ER negative disease. Several large studies on breast tumors have established at least two other major cores of amplification within the 11q13 locus, the GARP/D11S833E and D11S97/LOC91809 regions and in general, 11q13 amplifications may either involve amplification of a large region spanning all four cores, or a smaller region containing only one or a few cores. The CCND1 region is the most frequently amplified, constituting about two thirds of all 11q13 amplifications. Considering this complexity of amplification patterns, a more comprehensive mapping of the functional genes within this locus needs to be performed in order to elucidate whether CCND1-amplification is the primary event associated with an agonist effect of tamoxifen as implied in this study, or if it merely reflects the co-amplification of another, more crucial, gene and corresponding overexpression of a protein not yet identified. It can nevertheless be concluded that amplification of the CCND1-gene indeed seem to be an optimal marker for distinguishing tumors that have an adverse tamoxifen effect independent of the exact effect of other genes in the amplicon.

FISH-analyses of the CCND1 gene copy number is a fairly standardized technology that can be performed on formalin fixed material at centers today handling FISH-analyses of cerbB2. However, before we can implement our findings in the clinical setting, further retro- and prospective studies, also involving postmenopausal breast cancer patients, are urgently needed to validate and potentially confirm these alarming data. If this plausible negative effect of tamoxifen can be avoided in the future, we will be able to avoid disease progress and death in up to 15% of all breast cancer patients having a CCND1-amplified tumor. This would represent an enormous contribution to breast cancer treatment and opens up for the use of predictive markers in modern breast cancer therapy. The present study also clearly demonstrates that tamoxifen is an extremely efficient adjuvant treatment for non-CCND1-amplified ER-positive tumors and definitely has a role in future breast cancer treatment regimes, as well.

### FIGURE LEGENDS

FIG. 1. Examples of immunohistochemical staining of cyclin D1 (a=low and b=high), cyclin A2 (c=low and d=high) and fluorescent in situ analysis of CCND1 gene status (e-non-amplified and f=amplified)

FIG. 2. Recurrence free and overall survival for ER-positive cases with and without tamoxifen treatment according to cyclin A2 expression (a and c) and cyclin D1 expression (b and d).

FIG. 3. Recurrence free and overall survival for ER positive cases with and without treatment according to CCND1 gene status (A and B=all patients, C and D=lymoh node positive cases).

FIG. 4. Recurrence free survival and overall survival in women with amplified and non-amplified CCND1

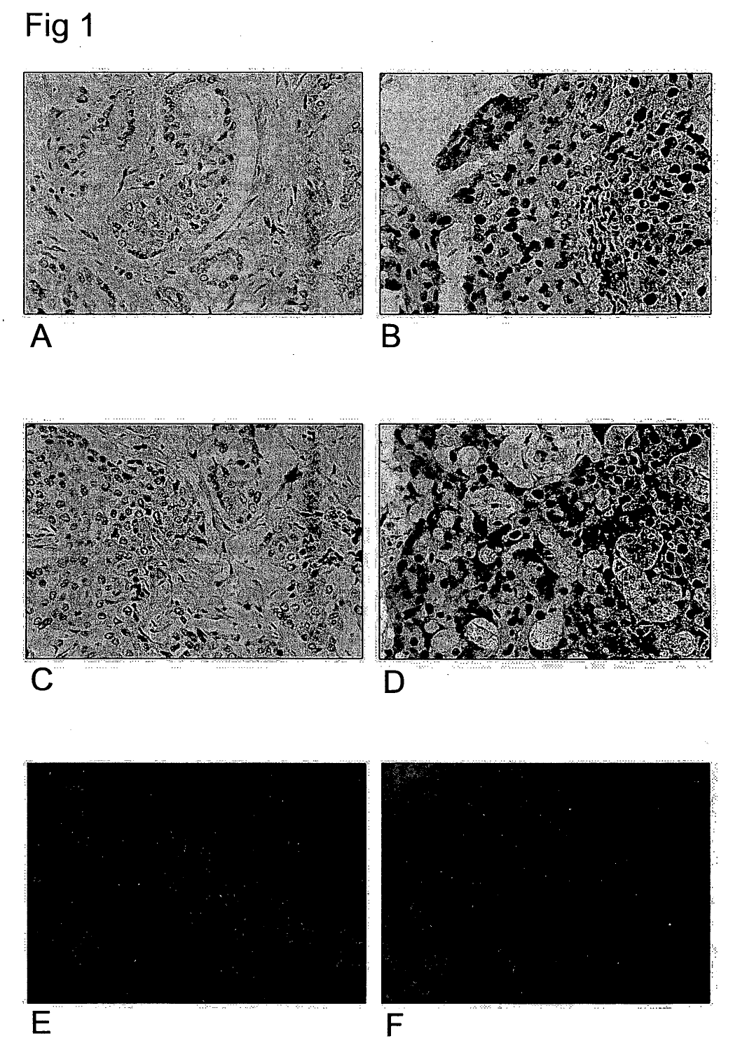

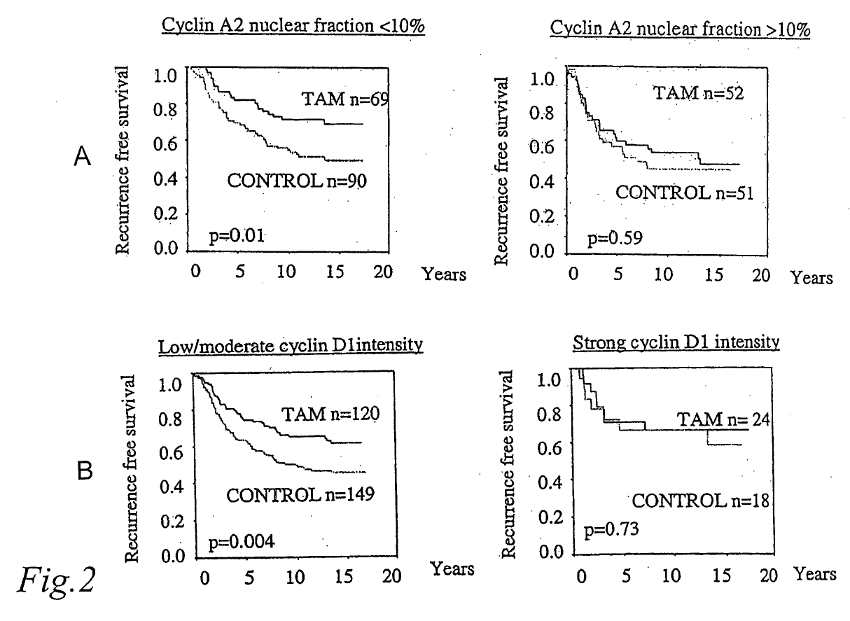

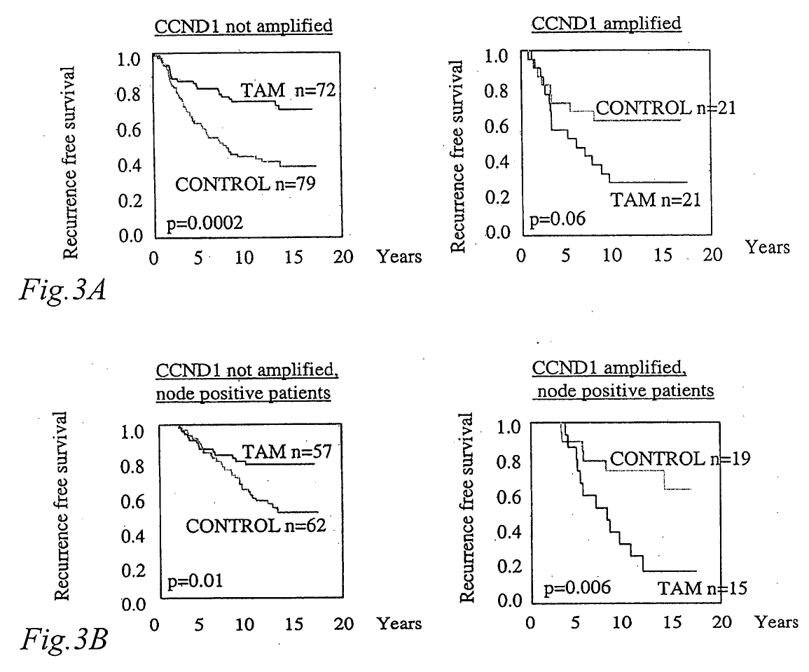

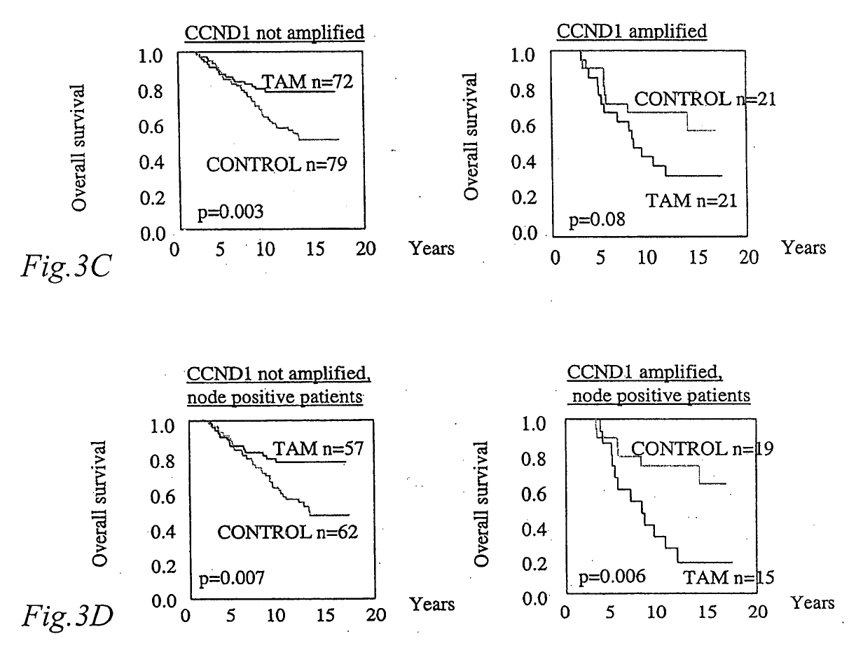

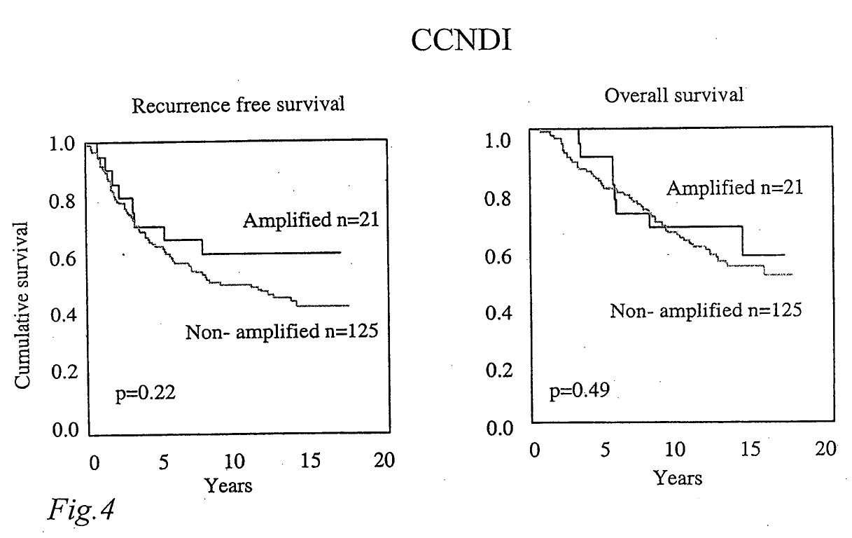

### REFERENCES

1. Buckley, M. F., et al., Expression and amplification of cyclin genes in human breast cancer. Oncogene, 1993. 8(8): p. 2127-33.
2. Keyomarsi, K. and A. B. Pardee, Redundant cyclin overexpression and gene amplification in breast cancer cells. Proc Natl Acad Sci USA, 1993. 90(3): p. 1112-6.
3. Gillett, C., et al., Amplification and overexpression of cyclin D1 in breast cancer detected by immunohistochemical staining. Cancer Res, 1994. 54(7): p. 1812-7.
4. Ormandy, C. J., et al., Cyclin D1, EMS1 and 11q13 amplification in breast cancer. Breast Cancer Res Treat, 2003. 78(3): p. 323-35.
5. Loden, M., et al., The cyclin D1 high and cyclin E high subgroups of breast cancer: separate pathways in tumorogenesis based on pattern of genetic aberrations and inactivation of the pRb node. Oncogene, 2002. 21(30): p. 4680-90.
6. Hui, R., et al., Cyclin D1 and estrogen receptor messenger RNA levels are positively correlated in primary breast cancer. Clin Cancer Res, 1996. 2(6): p. 923-8.
7. Cole, M. P., C. T. Jones, and I. D. Todd, A new anti-oestrogenic agent in late breast cancer. An early clinical appraisal of ICI46474. Br J Cancer, 1971. 25(2): p. 270-5.
8. Tamoxifen for early breast cancer: an overview of the randomised trials. Early Breast Cancer Trialists' Collaborative Group. Lancet, 1998. 351(9114): p. 1451-67.
9. Watts, C. K., et al., Antiestrogen inhibition of cell cycle progression in breast cancer cells in associated with inhibition of cyclin-dependent kinase activity and decreased retinoblastoma protein phosphorylation. Mol Endocrinol, 1995. 9(12): p. 1804-13.
10. Wilcken, N. R., et al., Inducible overexpression of cyclin D1 in breast cancer cells reverses the growth-inhibitory effects of antiestrogens. Clin Cancer Res, 1997. 3(6): p. 849-54.
11. Hodges, L. C., et al., Tamoxifen functions as a molecular agonist inducing cell cycle-associated genes in breast cancer cells. Mol Cancer Res, 2003. 1(4): p. 300-11.
12. Michalides, R., et al., Cyclin A is a prognostic indicator in early stage breast cancer with and without tamoxifen treatment. Br J Cancer, 2002. 86(3): p. 402-8.
13. Michalides, R., et al., A clinicopathological study on overexpression of cyclin D1 and of p53 in a series of 248 patients with operable breast cancer. Br J Cancer, 1996. 73(6): p. 728-34.
14. Rydén, L., et al., Two years of adjuvant tamoxifen in premenopausal patients with breast cancer: a randomized, controlled trial with long-term follow-up. Eur J Cancer, 2004. In Press.
15. Bukholm, I. R., G. Bukholm, and J. M. Nesland, Over-expression of cyclin A is highly associated with early relapse and reduced survival in patients with primary breast carcinomas. Int J Cancer, 2001. 93(2): p. 283-7.
16. Campbell, D. H., et al., Expression and tyrosine phosphorylation of EMS1 in human breast cancer cell lines. Int J Cancer, 1996. 68(4): p. 485-92.

1. A use of a method, whereby a tissue sample of a patient suffering from breast cancer and/or having been treated for breast cancer is tested with regard to amplification of the cyclin D1 gene, CCND1, wherein said amplification is a marker for the diagnosis of tamoxifen sensitivity.

2. A use according to claim 1, wherein estrogen positiveness or estrogen negativeness is determined, as well.

3. A use according to claim 1, wherein any node positiveness is determined, as well.
